SEQUENCE LISTING (1 sequence)

SEQ ID NO: 1 - DNA, 1489 bp, Artificial Sequence
  Synthetic gene derived from Sus scrofa aromatic amino acid decarb oxylase optimized for expression in E. coli
aattaaccatgggtaacgcttccgatttccgtcgtcgtggcaaagaaatggtagactacatggcagattatctggaaggtatcgaaggccgtcaagtttacccggacgttcagccaggctatctgcgtccgctcatcccagctaccgcaccgcaagaaccggacacctttgaagacatcctgcaagacgtagaaaagatcatcatgccaggtgtaacccactggcactctccgtactttttcgcatacttcccgactgcatcctcctacccggctatgctggctgacatgctgtgtggtgctatcggctgtatcggcttttcctgggctgcatctccggcatgcactgagctggaaaccgttatgatggattggctgggtaaaatgctgcagctgccagaggcatttctggctggtgaggctggtgagggtggtggtgtaattcaaggctctgcgtccgaagctactctggttgctctgctggctgctcgtactaaagttgttcgtcgtctgcaagctgcatctccgggtctgactcagggtgctgttctggagaaactggtagcgtatgcttctgatcaggctcactcttccgttgagcgtgctggtctgattggtggtgttaagctgaaagctattccgtccgatggtaagttcgctatgcgtgcatccgctctgcaagaagctctggaacgtgacaaagctgctggtctgattccgttcttcgttgttgctaccctgggtactacctcttgctgttctttcgacaacctgctggaagttggtccgatctgtcacgaggaggacatctggctgcacgttgacgcagcatatgctggctctgcttttatctgtccggaattccgtcacctgctgaacggcgttgagttcgctgattctttcaacttcaacccgcacaagtggctgctggttaactttgattgctcggctatgtgggtaaaacgtcgcactgatctgaccggtgcatttaaactggacccggtatatctgaagcattctcaccagggttccggcctgattaccgattatcgtcattggcagctgccgctgggtcgtcgttttcgttcgctgaagatgtggttcgtattccgtatgtacggcgttaaaggtctgcaagcatacatccgtaaacacgttcaactgtcgcacgagttcgaagctttcgtactgcaggacccgcgttttgaagtttgcgctgaagttaccctgggcctggtttgcttccgtctgaagggttctgatggtctgaacgaagctctgctggagcgtattaactcggctcgtaaaatccacctggttccgtgtcgtctgcgtggtcagttcgttctgcgcttcgctatttgttcgcgtaaggtagagtctggtcatgttcgtctggcatgggagcacatccgtggtctggctgctgaactgctggctgctgaagaaggtaaggctgaaatcaaatcctaatggatccagatcttcca

(19) United States
(12) Patent Application Publication
(10) Pub. No.: US 2003/0096379 A1
(43) Pub. Date: May 22, 2003

(54) Method for producing tryptamine derivatives

(76) Inventors: James L. Kilgore, Long Beach, CA (US); J. David Rozzell JR., Burbank, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/112,253

(22) Filed: Mar. 28, 2002

Related U.S. Application Data
(60) Provisional application No. 60/279,876, filed on Mar. 28, 2001.

(51) Int. Cl. C12P 13/22; C12P 17/10

(52) U.S. Cl. 435/108; 435/121

(57) ABSTRACT

The invention relates to a coupled enzymatic process for producing tryptamine derivatives from indole compounds. In the first enzyme-catalyzed reaction, indole derivatives are converted to tryptophan derivative intermediates, then the tryptophan intermediates are decarboxylated in a second enzymatic reaction in the same reaction system. In this way, tryptamine derivative products are formed from indole derivatives in a single process. The invention is also directed to novel tryptophan and tryptamine derivatives, which can be prepared by the inventive method.

### CROSS REFERENCE TO RELATED APPLICATION

This application claims priority of U.S. Provisional Application No. 60/279,876, filed Mar. 28, 2001, the entire disclosure of which is incorporated herein by reference.

### ACKNOWLEDGMENT OF GOVERNMENT SUPPORT

This invention was made with government support under Grant No. 1R43 MH62244, awarded by the National Institute of Health. The government has certain rights in the invention.

### FIELD OF THE INVENTION

This invention relates to novel methods for producing derivatives and to novel tryptophan and tryptamine derivatives.

### BACKGROUND

Tryptamine derivatives are of interest for use as neuropharmaceuticals and as biological probes for the study of neurologic phenomena. Tryptamine (indole-3-(2-ethane)amine, which is frequently referred to as TNH₂) and 5-hydroxytryptamine (serotonin) are primary neurotransmitter molecules and are therefore of fundamental importance in neurobiology. Functions of tryptamine derivatives include regulation of diurnal cycles, such as the onset of sleep and the modulation of fertility in humans and other mammals. Neuroactive tryptamine derivatives frequently bear one or more substituents on the amino nitrogen, as are found in N-acylindolethylamines such as melatonin and the N,N-dimethyl groups of the “triptan” migraine drugs, such as sumatriptan (Imitrex™), rizatriptan (Maxalt™), and zomitriptan (Zomig™).

Tryptamine derivatives may be produced by chemical synthesis, such as by attaching a 2-carbon chain to a previously prepared indole derivative (“aminoethylation”), by synthesizing an indole derivative that bears a substituent at the C-3 position that can later be converted into an ethylamino group, or by simultaneously forming an indole ring and attaching the ethylamino group to the indole ring. Each of these synthetic routes has drawbacks in terms of the substituents that can be introduced, the length and complexity of the synthesis, and the undesirable side products formed during the synthetic process.

A number of methods are available for “aminoethylation,” the attachment of a 2-carbon ethylamino group to an indole ring. Reaction of an indole with a strong base, such as a Grignard reagent, followed by reaction with aziridine directly gives the aminoethylated product in fair-to-moderate yields. Conjugate addition of an indole to nitroethylene followed by reduction of the nitro group achieves aminoethylation in two steps, although the reduction conditions for the nitro group are not compatible with certain other functional groups. Three-step procedures include the initial formation of an indole-3-carboxaldehyde, followed by condensation with nitromethane and subsequent reduction of both the double bond and nitro groups, as well as initial attachment of a (dimethylamino)methyl group to form a gramine derivative, followed by displacement with cyanide and reduction of the resulting nitrile to the amine.

All of the above-described synthetic routes to tryptamine and tryptamine derivatives suffer from known side reactions, resulting in reduced yield, wasted starting materials, and difficult purification steps. Aziridine adducts also have a tendency to oligomerize to produce polyethylenimine derivatives as a side reaction. Nitroethylene and other electron-deficient olefins participate in cycloaddition side-reactions with indole and its derivatives. The indole pyrrole ring itself is susceptible to reduction in the presence of acids, which are often added to reactions when cyano and nitro groups are reduced. De novo indole synthesis is also complicated by side reactions. For example, a substrate for the direct formation of sumatriptan by the Fischer indole synthesis has been shown to form a closely-related dimeric impurity comprising about 11% of the isolated products. Sumatriptan is the most widely-sold drug in its class, and a side reaction requiring careful chromatography to remove such an impurity would add significantly to the cost of a commercial process for its preparation.

### SUMMARY OF THE INVENTION

Because of the drawbacks inherent in the chemical syntheses of tryptamine derivatives, a synthetic route based on enzymatic catalysis offers an appealing alternative. Enzyme-catalyzed reactions are typically distinguished by mild reaction conditions, selectivity for the desired reaction, and few undesired side products. The present invention relates to an enzymatic route for the production of tryptamine derivatives that combines the action of two distinct enzymes. The combination of two enzymatic steps has the advantage of mild reaction conditions and few side reactions, leading to the efficient production of a wide range of different substituted tryptamines. In another embodiment, the present invention relates to novel substituted tryprophan and tryptamine compounds. These novel substituted tryptophan compounds can serve as key precursors for the production of the corresponding tryprtamines and also as key pharmaceutical intermediates. The novel tryptamine compounds are useful as intermediates for the production of neuroactive drugs and other bioactive molecules.

### DESCRIPTION OF THE DRAWING

These and other features and advantages of the present invention will be better understood by reference to the following detailed description when considered in conjunction with the accompanying figure wherein:

FIG. 1 is a DNA sequence of a synthetic gene derived from Sus scrofa aromatic amino acid decarboxylase optimized for expression in E. coli, wherein the underlined restriction sites are 5′-NcoI and 3′-BamHI.

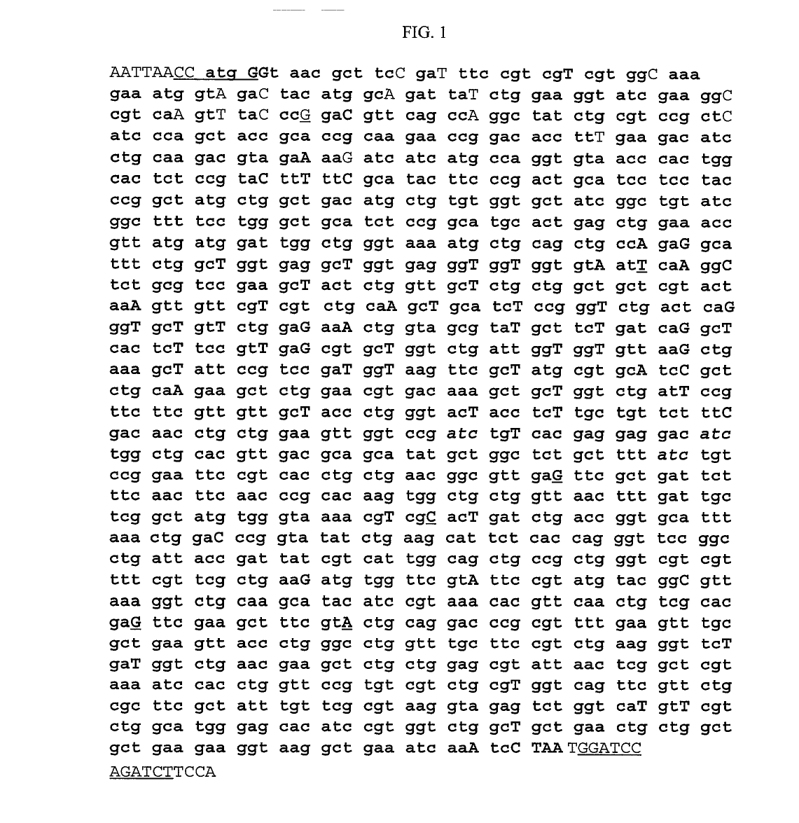

### DETAILED DESCRIPTION OF THE INVENTION

The present invention is directed to a novel method for preparing tryptamine derivatives as well as to novel tryptophan and tryptamine derivatives. In accordance with the method of the invention, an indole derivative, i.e., a substituted indole, is converted to an indole-3-(2-ethyl)amine by two enzymatic reactions. In the practice of this invention, two enzymes are used which, in combination, permit an efficient preparative process for tryptamine compounds bearing a wide range of substituents from inexpensive precursors, such as substituted indoles, pyruvic acid and ammonia.

The two enzymes used according to the inventive methods for the production of tryptamine derivatives are a tryptophan-synthesizing enzyme, which catalyzes the production of a substituted tryptophan from a substituted indole, and a tryptophan-decarboxylating enzyme, which catalyzes the conversion of a substituted tryptophan to the corresponding substituted tryptamine. The reactions catalyzed by the tryptophan-synthesizing enzyme and the tryptophan-decarboxylating enzyme can be carried out as separate reaction steps, with or without isolation of the intermediate substituted tryptophan, or both enzymes can be used together in a single reaction mixture to carry out the biocatalytic synthesis of a wide range of tryptamines.

As used herein, a tryptophan-synthesizing enzyme means any enzyme capable of catalyzing the synthesis of a substituted tryptophan from a substituted indole in combination with a precursor carboxylic acid having at least a 3-carbon chain that is α,α- or α,β-disubstituted with hetero atoms. Examples of tryptophan-synthesizing enzymes useful in the practice of this invention include enzymes under the EC number 4.2.1.20, such as tryptophan synthases, and enzymes under the EC number 4.1.99.1, such as tryptophanases.

As used herein, a tryptophan-decarboxylating enzyme means any enzyme capable of catalyzing the decarboxylation of a substituted tryptophan to produce the corresponding substituted tryptamine. Typical tryptophan-decarboxylating enzymes include enzymes from the family of aromatic amino acid decarboxylases (AAADs) and related enzymes. AAADs are ubiquitous enzymes, and can be isolated from animal tissues and plant sources in addition to various bacteria. Alternatively, a gene encoding a desired tryptophan-decarboxylating enzyme may be cloned into a suitable vector in an appropriate host organism. Sources of AAADs useful in the practice of this invention include pig kidney, rat brain, bovine brain, rat liver, and plants including Catharanthus roseus, Arabidopsis thalania, and Camptotheca acuminata. A large number of AAAD genes from insect sources, particularly Drosophila, have also been sequenced, and many of the corresponding enzymes have been cloned. Other examples of tryptophan-decarboxylating enzymes useful in the practice of this invention include enzymes under the EC number 4.1.1.28, such as tryptophan decarboxylases, DOPA decarboxylases, and other aromatic amino acid decarboxylases, as well as related enzymes that catalyze the decarboxylation of other similar amino acids, including tyrosine decarboxylases (EC 4.1.1.24), histidine decarboxylases (EC 4.1.1.22), phenylalanine decarboxylases (EC 4.1.1.53), and the like. Enzymes from these categories, as well as other decarboxylases, may be used to act on substituted tryptophans. One can easily determine whether the enzymes have the desired catalytic activity based on routine screening methods, discussed in the Examples below.

It is also possible to obtain appropriate tryptophan-synthesizing and tryptophan-decarboxylating enzymes useful in the practice of this invention by screening samples of microbial cultures or environmental samples. For example, diverse populations of enzymes can be found in microorganisms harvested from different environments. These microorganisms can be cultured, and their DNA extracted, amplified by PCR, and cloned into a host for expression of the enzymes. The enzymes can be recombinantly expressed, for example, in bacteria, in cultured cells of bacteria, fungi, or plants, or in a viral host.

Alternatively, tryptophan-synthesizing and tryptophan-decarboxylating enzymes useful in the practice of this invention can be obtain by the use of various molecular biology techniques, such as mutagenesis, shuffling, molecular breeding, and gene reassembly. These and related methods can be used to create vast numbers of mutant versions of an enzyme encoded by a known gene, and then the mutant enzymes can be screened for the desired catalytic activity. Examples of gene shuffling, gene reassembly, and molecular breeding are described in U.S. Pat. No. 5,605,793, U.S. Pat. No. 5,811,238, U.S. Pat. No. 5,830,721, U.S. Pat. No. 5,837,458, U.S. Pat. No. 5,965,408,. U.S. Pat. No. 5,958,672, U.S. Pat. No. 6,001,574, and U.S. Pat. No. 6,117,679, the disclosures of which are all incorporated herein by reference. Examples of methods for constructing large numbers of mutants are described in U.S. Pat. No. 6,001,574, U.S. Pat. No. 6,030,779, and U.S. Pat. No. 6,054,267, the disclosures of which are also incorporated herein by reference.

In one embodiment, one or both enzymes is a mutant produced by a random mutagenesis technique, such as error-prone PCR or treatment with mutagenic agents, such as, for example, alkyl sulfates, alkyl sulfonates and UV radiation. In another embodiment, one or both enzymes are mutants incorporating random alterations in a small defined region of the sequence that is constructed from synthetic oligonucleotides. In yet another embodiment, one or both enzymes are new enzymes produced by interchanging fragments of different, related enzymes (DNA shuffling).

A reaction illustrative of the inventive method is shown below:

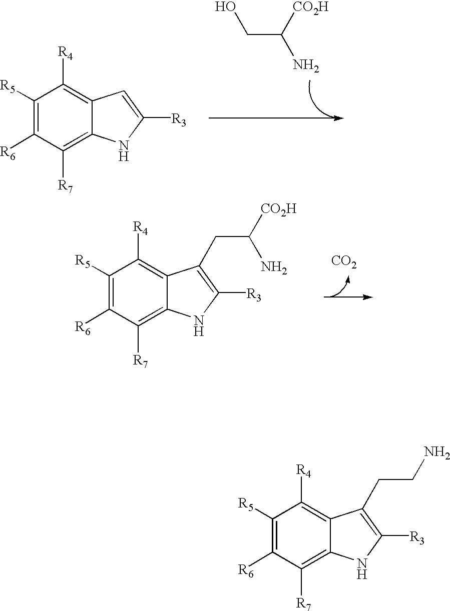

The first step of the inventive method involves contacting an indole derivative with a carboxylic acid having at least a 3-carbon chain that is α,α- or α,β-disubstituted with hetero atoms in the presence of a tryptophan synthase enzyme to produce a tryptophan derivative. Examples of such carboxylic acids include serine, pyruvate, 3-haloalanine, 3-acyloxyalanine, cysteine, S-alkylcysteines, S-acyl cysteines, threonine, and allothreonine. However, the structure of the carboxylic acid will vary depending on the desired final product.

The indole derivative is substituted in any one or more of the R₃ to R₇ positions, as shown in the above reaction scheme. The precise structure of the indole derivative is not critical to the invention. Preferred R₃ to R₇ substitutents include hydrogen, alkyl, alkenyl, alkynyl, cycloalkyl, cycloalkenyl, cycloalkylalkyl, cycloalkenylalkyl, aryl, aralkyl, heterocyclic rings, halo, hydroxy, alkoxy, carboxy, carboalkoxy, acyloxy, cyano, nitro, acyl, acyloxyalkyl, mercapto, thioalkyl, sulfonylalkyl, sulfenylalkyl, aminoacyl, sulfonylamino, N-methylsulfonylamino, and sulfinylalkyl, or two of R₄, R₅, and R₆ can together form a ring selected from the group consisting of cycloalkyl, cycloalkylalkyl, cycloalkenylalkyl, aryl, and heterocyclic rings. If two or more substituents are provided on the indole derivative, the substituents can be the same or different. In a particularly preferred embodiment, one of the R₄ to R₇ substituents is a lower (C₁ to C₄) alkyl group bearing a leaving group selected from OH, Cl, OS(═O)₂alkyl, OS(═O)₂aryl, O—S(═O)₂O⁻, O—P(═O)(O⁻)₂, O—P(═O)(O-aryl)₂, O—P(═O)(O-alkyl)₂, O—P(═O)(O-alkyl)₂, O—C(═NH)alkyl, O—C(═O)H, O—C(═O)alkyl, and O—C(═O)aryl.

Preferably the indole or indole derivative is contacted with β-substituted alanine derivative or pyruvate in the presence of a tryptophan-synthesizing enzyme, preferably a pyridoxal cofactor, and, in the case of α,α-disubstituted carboxylic components, preferably ammonia or ammonium ion, to give a tryptophan derivative. The tryptophan-synthesizing enzyme synthesizes a tryptophan derivative by adding an amino acid-containing side chain to the C-3 position of indole or an indole derivative. The indole derivative may be substituted in any position except for the C-3 position where the side chain is attached. Preferred indole derivatives contain substituents at the C-5 position of the indole ring, and a particularly preferred indole derivative contains a substituted methyl group at the C-5 position.

The precursor for the side chain group is an α-ketoacid or αβ-substituted alanine derivative with a chain length of three carbons or more, which is either an amino acid itself, or which can react with pyridoxal phosphate or a similar enzyme cofactor to give a reactive amino acrylic acid intermediate. Side chain precursors include L- and D-alanine derivatives in which the β-carbon is substituted with a heteroatom. The β-carbon may be disubstituted if desired. The term “β-substituted alanine derivative,” as used herein, refers to compounds of the formula:

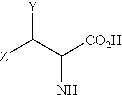

wherein Y is selected from the group consisting of hydroxy, halo, sulfhydryl, alkylthio, phosphoryloxy, acyloxy and alkoxy, and Z is selected from the group consisting of hydrogen, alkyl, alkenyl, alkynyl, cycloalkyl, cycloalkenyl, cycloalkylalkyl, cycloalkenylalkyl, aryl, aralkyl, heterocyclic rings, carboxy, cyano, nitro, acyl, acyloxyalkyl, sulfonylalkyl, sulfenylalkyl, and sulfinylalkyl. Thus the natural substrate serine may be replaced with other amino acids that bear groups that can be eliminated concurrent with loss of the a-hydrogen, such as O-substituted serines, β-halo alanines, cysteine, S-substituted cysteine, threonine, and 3-halogen-substituted amino acids. Preferred sidechain precursors include serine or pyruvic acid in the presence of an ammonium ion source, such as ammonia or ammonium ion. In a particularly preferred embodiment, the sidechain precursor is L-serine, either in pure form or as the racemate, wherein the D-isomer is racemized in the reaction mixture by a third enzyme, amino acid racemase, or serine racemase. Preferred carboxylic acids include D-serine, L-serine, O-alkyl derivatives of serine, O-acyl derivatives of serine, L-cysteine, S-alkyl derivatives of cysteine, S-acyl derivatives of cysteine, 3-halo-L-alanine derivatives, α-amino acids with a 4-carbon or longer alkyl chain that is substituted on the β-position with an oxygen, sulfur or halogen leaving group, and salts thereof.

The second step of the method is the decarboxylation of the tryptophan derivative in the presence of a tryptophan-decarboxylating enzyme to produce a tryptamine derivative. The tryptophan-decarboxylating enzyme is, as aforementioned, any enzyme capable of decarboxylating the tryptophan intermediate to produce a corresponding tryptamine. In accordance with the inventive method, tryptophan derivatives are decarboxylated to form tryptophamine derivatives in high yields.

The tryptophan-synthesizing enzyme and tryptophan-decarboxylating enzyme are used, as aforementioned, to carry out two consecutive reactions converting a substituted indole to a correspondingly substituted tryptamine. In one embodiment the tryptophan-synthesizing enzyme and tryptophan-decarboxylating enzyme carry out their respective reactions in separate steps with or without isolation of the substituted tryptophan intermediate. In another embodiment, the two enzymes maybe used to carry out their respective reactions in a single-pot reaction without the need for isolation of the substituted tryptophan intermediate. If desired, one or both of the enzymes is separated from the product solution by means of a physical attachment or barrier. For example, one or both enzymes may be separated from the product solution by a porous membrane for retaining high molecular weight compounds. One or both of the enzyme catalysts may be immobilized on a solid support through covalent bonds, through a strong noncovalent physical adsorption mechanism, or through ionic bonding, or one or both of the enzyme catalysts may be adsorbed on a solid support through nonpolar, hydrophobic bonding. The immobilized enzyme(s) maybe used in a flow-reactor system. The tryptamine derivative produced may be isolated from the enzyme reaction mixture by chemoselective adsorption onto a solid surface. The adsorbing surface is preferably a nonpolar material composed of an alkyl, aryl, heterocyclic ring or similar hydrophobic material. Preferably the adsorbing surface bears anionic groups selected from sulfonates, carboxylates, borates, boronates, phosphates, and phosphonates.

The tryptamine products may be further elaborated by known chemical means to provide biologically active products. Common substituents include N_{β}-methyl and N-acetyl groups, as well as saturated carbocyclic and heterocyclic rings.

Using the method of the present invention, an appropriately substituted indole can be converted to substituted tryptophan in a first enzymatic step using a suitable tryptophan-synthesizing enzyme, and the substituted tryptophan is subsequently decarboxylated to produce the corresponding tryptamine. The decarboxylation of the substituted tryptophan is carried out in the presence of a tryptophan-decarboxylating enzyme. Although chemical methods have been developed for decarboxylation of tryptophans, these methods require high temperatures (180-200° C.). Such harsh reaction conditions are not tolerated by certain substituents of interest in drug development. In contrast, decarboxylation catalyzed by a suitable tryptophan-decarboxylating enzyme can be carried out under mild conditions and lower temperatures.

Preferably, both of the enzymatic reactions of the present invention are carried out at a temperature ranging from about 15° C. to about 95° C., and more preferably at a temperature ranging from about 25° C. to about 75° C. The enzymatic reactions of the present invention are preferably carried out in aqueous or predominantly aqueous conditions. By the term “predominantly aqueous conditions” is meant a solution that contains about 50% or more water by volume. Optionally, non-aqueous additives may also be present. Example of such non-aqueous additives include water-miscible solvents such as methanol, ethanol, isopropanol, acetone, acetonitrile, dimethyl formamide, dimethyl sulfoxide, polyethylene glycol, and the like. Water soluble carbohydrates, including glucose, sucrose, galactose, lactose, trehelose, and the like, may also be used as additives. Other additives include salts such as sodium chloride, potassium chloride, ammonium sulfate, and the like. Water-immiscible solvents can also be used as additives in the practice of the present invention. Such water-immiscible solvents include toluene, heptane, ethyl acetate, butyl acetate, methyl t-butyl ether, and the like.

In one embodiment, the indole substrate is dissolved in an organic solvent that is in contact with an aqueous solution of the carboxylic acid and other reaction components. The organic solvent is preferably selected from alkyl ethers, aryl ethers, aromatic hydrocarbons, aliphatic alcohols, alkyl esters, aliphatic nitrites and halogenated hydrocarbons.

The coupled enzyme reactions of the invention have significant advantages over single reaction processes. A second enzyme may regenerate a cofactor consumed in the reaction or remove a product formed in an enzyme-catalyzed equilibrium, thereby making the initial reaction irreversible. Tryptophan derivatives are both amphoteric and hydrophobic compounds, and are therefore more difficult to purify than tryptamine derivatives, which behave as “simple” amines. In aqueous-organic separations, tryptamines may be extracted into either aqueous acid or organic solvents depending on the pH of the aqueous phase. Furthermore, a coupled enzyme process offers speed and simplicity as compared to multi-step chemical processes. However, it should be noted that it is possible to synthesize and purify tryptophan derivatives in a single enzyme reaction.

The present invention is also directed to novel tryptophan and tryptamine derivatives, which are preferably produced in accordance with the above-described methods. These compounds include tryptophans and tryptamines with substituents at various positions on the carbon skeleton. The compounds of the invention have the Formula I:

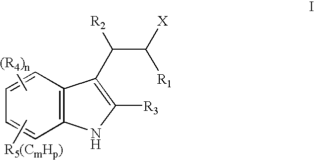

wherein:

X is hydrogen or CO₂H;

R₁ is selected from the group consisting of heterocyclic rings containing nitrogen and NR₈R₉, wherein R₈ and R₉ are each independently selected from the group consisting of hydrogen, alkyl, cycloalkyl, heterocycloalkyl, CO-R₁₀, and NHC(O)—R₁₀, wherein R₁₀ is selected from the group consisting of hydrogen, alkyl, alkoxy, cycloalkyl, aryl, and heterocyclic rings;

R₂ and R₃ are each independently selected from the group consisting of hydrogen, alkyl, alkenyl, alkynyl, cycloalkyl, cycloalkenyl, cycloalkylalkyl, cycloalkenylalkyl, aryl, aralkyl, heterocyclic rings, halo, hydroxy, alkoxy, carboxy, carboalkoxy, acyloxy, cyano, nitro, acyl, acyloxyalkyl, mercapto, thioalkyl, sulfonylalkyl, sulfenylalkyl, aminoacyl, sulfonylamino, N-methylsulfonylamino, and sulfinylalkyl, or two groups together form a ring selected from the group consisting of cycloalkyl, cycloalkylalkyl, cycloalkenylalkyl, aryl, and heterocyclic rings;

each R₄ group is independently selected from the group consisting of alkyl, alkenyl, alkynyl, cycloalkyl, cycloalkenyl, cycloalkylalkyl, cycloalkenylalkyl, aryl, aralkyl, heterocyclic rings, halo, hydroxy, alkoxy, carboxy, carboalkoxy, acyloxy, cyano, nitro, acyl, acyloxyalkyl, mercapto, thioalkyl, sulfonylalkyl, sulfenylalkyl, aminoacyl, sulfonylamino, N-methylsulfonylamino, and sulfinylalkyl, or two R₄ groups together form a ring selected from the group consisting of cycloalkyl, cycloalkylalkyl, cycloalkenylalkyl, aryl, and heterocyclic rings;

R₅ is a leaving group selected from the group consisting of OH, Cl, OS(═O)₂alkyl (preferably OS(═O)₂perfluoroalkyl), OS(═O)₂aryl, O—S(═O)₂O⁻, O—P(═O)(O⁻)2, O—P(═O)(O-aryl)₂, O—P(═O)(O—alkyl)₂, O—P(═O)(O-alkyl)₂, O—C(═NH)alkyl (preferably O—C(═NH)CCl₃), O—C(═O)H, O—C(═O)alkyl (preferably O—C(═O)perchloroalkyl or O—C(═O)perfluoroalkyl), and O—C(═O)aryl (preferably O—C(═O)-4-NO₂Ph);

m ranges from 1 to 4;

n ranges from 0 to 3; and

p equals 2m or 2m−2;

wherein, when X is CO₂H, two R₄ groups do not together form a ring.

As used herein, the term “alkyl,” alone or in combination, means a straight-chain or branched-chain alkyl group containing from 1 to about 18 carbon atoms. Any of the carbon atoms may be substituted with one or more substituents selected from the group consisting of hydroxy, alkoxy, acyloxy, acylamido, halo, nitro, sulfhydryl, sulfide, thio, carboxyl, oxo, seleno, phosphate, phosphonate, phosphine, and the like. Examples of such alkyl groups include methyl, ethyl, chloroethyl, propyl, isopropyl, butyl, isobutyl, tertiary-butyl, 3-fluorobutyl, 4-nitrobutyl, 2,4-dibromobutyl, pentyl, isopentyl, neopentyl, 3-ketopentyl, hexyl, 4-acetamidohexyl, 3-phosphonoisohexyl, 4-fluoro-5,5-dimethylpentyl, 5-phosphinoheptyl, octyl, nonyl dodecyl, and the like.

As used herein, the term “alkenyl,” alone or in combination, means a straight-chain or branched-chain hydrocarbon group containing one or more carbon-carbon double bonds and containing from 2 to about 18 carbon atoms. Any of the carbon atoms maybe substituted with one or more substituents selected from hydroxy, alkoxy, acyloxy, acylamido, halo, nitro, sulfhydryl, sulfide, carboxyl, oxo, seleno, phosphate, phosphonate, phosphine, and the like. Examples of such alkenyl groups include ethenyl, propenyl, allyl, 1,4-butadienyl, 2-pentenyl, 3-pentenyl, 4-pentenyl, 2,6-decadienyl, 2-fluoropropenyl, 2-methoxypropenyl, 2-carboxypropenyl, 3-chlorobutadienyl, and the like.

As utilized herein, the term “alkynyl,” alone or in combination, means a straight-chain or branched-chain hydrocarbon group containing one or more carbon-carbon triple bonds and containing from 2 to about 18 carbon atoms. Any of the carbon atoms may be substituted with one or more substituents selected from the group consisting of alkoxy, acyloxy, acylamido, halogen, nitro, sulfhydryl, sulfide, carboxyl, oxo, seleno, phosphate, phosphonate, phosphine, and the like. Examples of such alkynyl groups include ethynyl, propynyl, 1,4-butadiynyl, 3-pentynyl, 2,6-decadiynyl, 2-fluoropropynyl, 3-methoxy-1-propynyl, 3-carboxy-2-propynyl, 3-chlorobutadiynyl, and the like.

As utilized herein, the term “cycloalkyl,” alone or in combination, means an alkyl group which contains from about 3 to about 12 carbon atoms and is cyclic. Any of the carbon atoms may be substituted with one or more substituents selected from the group consisting of hydroxy, alkoxy, acyloxy, acylamido, halo, nitro, sulfhydryl, sulfide, carboxyl, oxo, seleno, phosphate, phosphonate, phosphine, and the like. Examples of cycloalkyl groups include cyclopropyl, cyclobutyl, cyclopentyl, cyclohexyl, cycloheptyl, 2-methylcyclopentyl, 3-methylcyclohexyl, various substituted derivatives, and the like.

As utilized herein, the term “cycloalkenyl,” alone or in combination, means an alkenyl group which contains from about 3 to about 12 carbon atoms and is cyclic. Any of the carbon atoms may be substituted with one or more substituents selected from the group consisting of alkoxy, acyloxy, acylamido, halo, nitro, sulfhydryl, alkylthio-, carboxyl, oxo, seleno, phosphate, phosphonate, phosphine, and the like. Examples of cycloalkyl groups include cyclopropenyl, cyclobutenyl, cyclopentenyl, cyclohexenyl, cyclohexadienyl, cycloheptenyl, 2-methylcyclopentenyl, 3-methylcyclohexenyl, 3-chlorocyclohexenyl, 3-carboxymethylcyclopentenyl, and the like.

As used herein, the term “cycloalkylalkyl,” alone or in combination, means an alkyl group as defined above which is substituted by a cycloalkyl group containing from about 3 to about 12 carbon atoms. Any of the carbon atoms may be substituted with one or more substituents selected from the group consisting of hydroxy, alkoxy, acyloxy, acylamido, halo, nitro, sulfhydryl, sulfide, carboxyl, oxo, seleno, phosphate, phosphonate, phosphine, and the like. Examples of cycloalkyl groups include cyclopropyl, cyclobutyl, cyclopentyl, cyclohexyl, cycloheptyl, 2-methylcyclopentyl, 3-methylcyclohexyl, 3-fluoromethylcyclohexyl, 3-carboxymethylcyclohexyl, 2-chloro-3-methylcyclopentyl, and the like.

As used herein, the term “cycloalkenylalkyl,” alone or in combination, means an alkyl group as defined above which is substituted with a cycloalkenyl group containing from about 3 to about 12 carbon atoms. Any of the carbon atoms may be substituted with one or more substituents selected from the group consisting of alkoxy, acyloxy, acylamido, halo, nitro, sulfhydryl, sulfide, carboxyl, oxo, seleno, phosphate, phosphonate, phosphine, and the like. Examples of cycloalkenylalkyl groups include cyclopropenyl, cyclobutenyl, cyclopentenyl, cyclohexenyl, cycloheptenyl, 2-methylcyclopentenyl, 3-methylcyclohexenyl, 3-fluoromethylcyclohexenyl, 3-carboxymethylcyclohexenyl, 2-chloro-3-methylcyclopenentyl, 3-nitrocyclohexenyl, and the like.

As used herein, the term “aryl,” alone or in combination, means a carbocyclic aromatic system containing 1, 2, or 3 rings, wherein such rings may be attached in a pendent manner to each other or may be fused to each other. Examples of aryl groups include phenyl, naphthyl, biphenyl, and the like, which may optionally be substituted at any available ring position with one or more substituents selected from the group consisting of hydroxy, alkoxy, acyloxy, acylamido, halo, nitro, sulfhydryl, sulfide, carboxyl, oxo, seleno, phosphate, phosphonate, phosphine, and the like. Examples of such aryl groups include phenyl, 4-fluorophenyl, 2-chloroethyl, 3-propylphenyl, 1-naphthyl, 2-naphthyl, 2-methoxy-1-naphthyl, 3,4-dimethoxyphenyl, 2,4-difluorophenyl, and the like.

As utilized herein, the term “aralkyl,” alone or in combination, means an alkyl group as defined above which is substituted with an aryl group as defined above. Examples of aralkyl groups include benzyl, 2-phenylethyl, 2,4-dimethoxybenzyl, 4-fluorobenzyl, 4-chlorobenzyl, 4-bromobenzyl, 4-iodobenzyl, m-hydroxy-3-phenylpropyl, 2-(2-naphthyl)ethyl and the like.

As used herein, the term “heterocyclic ring,” alone or in combination, means a saturated, unsaturated or partially unsaturated monocyclic, bicyclic, or tricyclic group containing one or more heteroatoms as ring atoms, said heteroatoms selected from oxygen nitrogen, sulfur, phosphorous, selenium, and silicon. Any of the carbon atoms in the heterocyclic ring may be optionally substituted with one or more substituents selected from the group consisting of hydroxy, alkoxy, acyloxy, acylamido, halo, nitro, sulfhydryl, sulfide, carboxyl, oxo, seleno, phosphate, phosphonate, phosphine, and the like. Examples of such heterocyclic rings include imidazoyl, oxazolinyl, piperazinyl, pyrrolidinyl, phthalimidoyl, maleimidyl, thiamorpholinyl, various substituted derivatives, and the like.

As used herein, the term “alkoxy,” alone or in combination, means a group of the general formula —OR wherein R is a group selected from alkyl, alkeneyl, alkynyl, cycloalkyl, cycloalkenyl, cycloalkylalkyl, cycloalkenylalkyl, aryl, aralkyl, heterocyclic rings, and the like.

As used herein, the term “carboxy,” alone or in combination, means a group having a carbonyl group, such as a carboxylic acid, a ketone, an ester, and the like.

As used herein, the term “thioalkyl,” alone or in combination, means a group of the general formula —SR wherein R is a group selected from alkyl, alkeneyl, alkynyl, cycloalkyl, cycloalkenyl, cycloalkylalkyl, cycloalkenylalkyl, aryl, aralkyl, heterocyclic rings, and the like.

As used herein, the term “sulfonylalkyl,” alone or in combination, means a group of the general formula —S(O)₂R wherein R is an alkyl group.

As used herein, the term “sulfenylalkyl,” alone or in combination, means a group of the general formula —SOR wherein R is an alkyl group.

As used herein, the term “aminoacyl,” alone or in combination, means a group of the general formula —C(O)NRR′ wherein R and R′ are each a group independently selected from hydrogen, alkyl, alkeneyl, alkynyl, cycloalkyl, cycloalkenyl, cycloalkylalkyl, cycloalkenylalkyl, aryl, aralkyl, heterocyclic rings, and the like.

As used herein, the term “sulfonylamino,” alone or in combination, means a group of the general formula —S(O)₂NRR′ wherein R and R′ are each a group independently selected from hydrogen, alkyl, alkeneyl, alkynyl, cycloalkyl, cycloalkenyl, cycloalkylalkyl, cycloalkenylalkyl, aryl, aralkyl, heterocyclic rings, and the like.

As used herein, the term “sulfinylalkyl,” alone or in combination, means a group of the general formula —S(O)OR wherein R is an alkyl group.

Particularly preferred compounds include those where R₁ is NH₂ or N(CH₃)₂ and R₅ is selected from OH, OSO₂CH₃, OSO₂C₆H₅CH₃, OSO₂CF₃, and OSO₂C₆H₅NO₂. Hydroxymethyl tryptamine is of particular interest because it can be used as a precursor to the synthesis of the migrane drug sumitriptan. Once hydroxymethyl tryptamine is synthesized, conversion to sumitriptan can be accomplished easily by aminosulfonation.

The invention will now be described by the following examples, which are presented here for illustrative purposes only and are not intended to limit the scope of the invention.

### EXAMPLES

### Example 1

Tryptophan-Synthesizing Enzymes

Genes from wild-type E. coli DH5α tryptophan synthase (TrpBA [E.C.4.2.1.20]) and tryptophanase [TnA E.C. 4.1.99.1]) were cloned in the laboratory and were expressed in either E. coli LMG or E. coli BL-21 using the pBAD and pET vectors, respectively. Native TrpBA exists as an α₂β₂-tetramer. (See Miles. E. W. et al, Methods Enzymol., 1987, 142, 398-414, hereby incorporated by reference.) Genes from the native E. coli DNA sequence were cloned in series in a single plasmid in order to coexpress both α- and β-subunits. (See Das, A.; Yanofsky, C. Nuel. Acids. Res., 1989, 17,933-9340, hereby incorporated by reference.) The α-subunit, whose catalytic function is to produce indole from indoleglycerol phosphate is included because it is known that the heteroenzyme complex is more active for tryptophan synthesis from indole than the isolated β-subunit. (See Miles, E. W., et al., Methods Enzymol., 1987, 142,398-414, and Ogashara, K et al., J. Biol. Chem., 1992, 267, 5222-5228, both of which are hereby incorporated by reference.) For the TrpBA complex, the denaturing PAGE band corresponding to the α-subunit was substantially weaker in both crude and chromatographed preparations, so expression levels of the two subunits were most likely unequal, giving rise to an enzyme preparation containing a mixture of α₂β₂-tetramer and β₂-homodimer. (See Ahmed, S. A. et al., Biochemistry, 1987, 26, 5492-5498, which is incorporated herein by reference.) The gene for E. coli TnA was obtained by PCR by using the published sequence. (See Deely, M. C.; Yanofsky, C. J. Bacteriol., 1981, 147, 787-796, which is incorporated herein by reference.)

### Example 2

Decarboxylase Enzymes

Pig kidney tissue was quick-frozen in liquid N₂ prior to storage at −80° C. prior to extraction of the enzyme aromatic amino acid decarboxylase (pkAAAD [E.C. 4.1.1.28]). Frozen tissue was partially thawed in lysis buffer, then it was extracted and partially purified as described in Dominici, P.; Moore, P. S.; Voltattomi, C. B. Protein, Purif. Expr., 1993, 4, 345-347, which is incorporated herein by reference, omitting the phenyl-Sepharose chromatography. The gene for pkAAAD was cloned but poorly expressed. (See Moore, P. S.; Dominici, P.; Voltattorni, C. B. Biochem. J., 1996, 315, 249-256, which is incorporated herein by reference.) A synthetic gene (synAAAD, SEQ. ID 1), which was optimized for protein expression in E. coli (See Bui, P. and Rozzell, J. D., 2000, which is incorporated herein by reference), based on the published protein sequence of pkAAAD (See Maras, B. et al., Eur. J. Biochem., 1991, 201, 385-391, which is incorporated herein by reference). One DEAE-cellulose chromatography gave sufficiently pure enzyme for substrate screening. Plasmids containing the C. accuminata genes for tryptophan decarboxylases (TDC1 and TDC2) in the pKK233 expression vector are described by C. Nessler. (See Lopez-Meyer, M.; Nessler, C. L. Plant J., 1997, 11, 1167-1175, which is incorporated herein by reference.) These plasmids were transferred directly into E. coli DH5α cells for enzyme expression.

### Example 3

Enzyme Cloning and Expression

E. coli-derived enzymes were cloned into pBAD or pET expression vectors (Invitrogen) and expressed in LMG194 or BL-21 E. coli strains, respectively. TDC 1 and TDC2 plasmids in pKK233 vectors were propagated and expressed in E. coli DH5α TnaA and TrpBA genes were amplified by using E. coli DH5α chromosomal DNA as the template with slight modifications to insert restriction sites and improve expression. SynAAAD was cloned into a pET-15 vector. Enzyme expression was induced by L-arabinose (pBAD) or IPTG (pET, pk233) during growth in LB medium (See Sambrook, J.; Fritsch, E. F.; Maniatis, T. Molecular Cloning, A Laboratory Manual, Cold Spring Harbor Laboratory Press, Cold Spring Harbor, N.Y. 2001). Denaturing PAGE was performed using Novex NuPAGE MES SDS gels and running buffers. Protein in solution was measured by the Bradford procedure (Bradford, M. Anal. Biochem. 1976).

### Example 4

Enzyme Extraction and Purification

General Procedure:

All buffers contained 0.1 mM each DTT and PLP; extraction buffers for AAADs and TDCs also contained the protease inhibitors 0.5 mM α-phenylmethanesulfonyl fluoride, 1 μM leupeptin and 0.1-μM pepstatin.

Cells were harvested by centrifugation and were resuspended in 7 ml/g wet weight of extraction buffer. The resulting suspension was passed once through an Avestin Emulsiflex C-5 cell disruptor, operating at a maximum pressure of 10,000-12,000 psi. Following centrifugation of the resulting cell homogenate for 20 min at 14,000×g in a Sorvall GS/A rotor, the supernatant was dialyzed overnight against a single change of 10 mM pH 7.0 sodium phosphate (KPi) buffer. The dialysate was concentrated to approximately one-third of the original volume by placing the dialysis bag in a beaker containing flakes of polyethylene glycol (PEG-8000 avg. MW 8000) over a period of 2 to 4 h. The clarified concentrate was applied to a column of ion-exchange cellulose (Whatman DE52) equilibrated in 10 mM KPi buffer pH 7.5, and the column was then washed with 10 mM KPi, followed by higher concentration elution buffer (50-100 mM KPi). Active fractions were pooled and concentrated using PEG-8000 as before and the concentrates were dialyzed against 10 mM pH 7.5 KPi.

### Example 5

General Assay Methodology and Analytical Techniques

Tryptophan synthase activity was assayed by monitoring the increase in absorbance at 290 nm of a mixture containing serine, indole and PLP (Higgins, W., et al., Biochemistry, 1979, 18, 4827). TnA-catalyzed tryptophan synthesis was also measured by the 290 nm assay, while the tryptophan lyase reaction rate was measured by monitoring the reduction in NADH in reaction mixtures containing L-tryptophan and lactate dehydrogenase, indicating the amount of pyruvate released. Tryptophan decarboxylase was assayed colorimetrically by monitoring the sabsorbance at 580 nm produced by serotonin (AAADs) or tryptamine (TDCs) following treatment of reaction aliquots with Ehrlich's reagent (4-N,N-(dimethyl)aminobenzaldehyde in ethanolic HCl; Nakazawa, H.; Kumagi, H.; Yamada, H. Biochem Biophys. Res. Commun., 1974, 61, 75-82). Serotonin could be detected with approximately 5-fold higher sensitivity by this method as compared with tryptamine. The Ehrlich reagent assay using a microtiter plate format for simultaneous analysis of multiple enzyme samples may also be used. HPLC analysis was performed on acid-quenched reaction aliquots by using a base-deactivated C8 reversed-phase support (Shandon Hypersil BD8). HPLC analysis of acid-quenched aliquots permitted calculation of a rate ratio of 4.5 to 1 for pkAAAD-catalyzed DOPA decarboxylation relative to that of L-5-hydroxytryptophan, in good agreement with the accepted ratio of approximately 5:1. (See Sourkes, T. L. Methods Enzymol., 1987, 142, 170-178, which is incorporated herein by reference.)

Enzyme Activity Units

All activities are expressed in units of μmol/min enzyme product formed. Comparison of these results with literature values requires conversion of units as follows:

AAAD: One 5-hydroxytryptophan decarboxylase unit=5000 DOPA decarboxylase units (Voltattomi, C. B., et al., Methods Enzymol., 1987, 142, 179-187); TrpBA: One tryptophan synthase unit=10 literature units (Miles, E. W., et al., Methods Enzymol., 1987, 142, 398-414).

HPLC was used to detect indoles, tryptophans and tryptamines on a deactivated C8 stationary phase (Hypersil DB8). Reaction rates were estimated by HPLC analysis of aliquots quenched 1:3 v/v with 0.45 M sodium citrate buffer in 1:9 v/v acetonitrile/water. Electrospray mass spectral analysis of tryptophan and tryptamine products of the enzyme reactions were performed on samples collected under analytical HPLC conditions and on ion-exchange purified products.

### Example 6

Qualitative TrpBA and TnA Tryptophan Synthesis Assay by TLC

A mixture containing 2 μmole of the indole derivative, 0.05 μmole of PLP and 50 μmol of L-serine in 40 mM KPi buffer in 8% v/v ethanol water, pH 7.5, was warmed to 37° C., then enzyme solution was added to give a final volume of 1 mL. Aliquots (100 μL) were quenched at intervals of 2, 6, and 24 hours or more in microcentrifuge capsules containing 25 μL of 6 M HCl. The quenched samples were evaporated in a Savant Speed-vac. The residues were resuspended in methanol/water (1:1, 50 μL) and analyzed by TLC on Silica gel GF₂₅₄ plates alongside tryptophan and tryptamine standards, eluting with CH₂Cl₂/EtOH/H₂O/HOAc 77:20:2:1. Products were visualized first by using short-wave UV light, then with ninhydrin spray reagent (1/1/98 w/v/v ninhydrin/HOAc/EtOH) followed by heating.

### Example 7

Quantitative Assay for Tryptophan Synthesis at 290 nm (Higgins, W., et al, Biochemistry, 1979, 18, 4827)

Reaction mixtures were prepared as in Example 6 in polymethacrylate or quartz spectrophotometer cuvettes, and the increase absorbance were measured immediately at 290 nm, assuming a change in extinction coefficient for the indole-tryptophan conversion is δ₂₉₀=1.85 MM₋₁ cm⁻¹. For TrpBA, the rate at 37° C. is approximately 2.8 fold higher than at 23° C.

### Example 8

Quantitative Tryptophan Synthesis Assay by HPLC

Reaction mixtures were prepared as in Example 6 and were incubated at 37° C. Aliquots (100 μL) were removed and quenched by addition of to 300 μL of 0.5 M sodium citrate, pH 3.0 in 10% v/v acetonitrile/water in 2 ml microcentrifuge tubes. Following centrifugation for 10 min at 13,000×g, the supernatants were passed through 0.45μ membrane filters prior to HPLC analysis of 20 μL of filtrate.

HPLC conditions:

Column: Hypersil C8 BDS 5μ4.6×150 mm. Mobile phase: A-0.1% trifluoroacetic acid/water B-0.1% trifluoroacetic acid/acetonitrile Flowrate 1.0 mL/min

Time program: 0-2 min 0% B; 2-4 min 0-30% B; 4-10 min 30% B; 10-11 min 30-80% B; 11-14 min 80% B; 14-16 min 80-0% B. Detection: UV 280 nm

### Example 9

Qualitative AAAD Assay by TLC

A mixture containing 1 μmole of the tryptophan derivative, 0.05 μmole of PLP and 50 μmol of L-serine in 40 mM pH 7.5 KPi buffer was warmed to 37° C., then enzyme solution was added to give a final volume of 1 mL. Aliquots (100 HL) were quenched at intervals and were evaporated in a Savant Speed-vac as in Example 6. For TLC analysis, CH₂Cl₂/EtOH/H₂O/HOAc 49:49:1:1 was used as the solvent. The plates were visualized with short-wave UV light and ninhydrin as in Example 6.

### Example 10

1 mL Scale 5-HOtryptophan Decarboxylase Assay Using Ehrlich 's Reagent

A mixture (0.95 mL) containing L-5-hydroxytryptophan (2 mM), PLP (0.25 mM) and pH 7.5 KPi buffer (44 mM) was warmed to 37° C., then 50 μL of enzyme was added. Reaction aliquots were quenched in microcentrifuge capsules containing 50 μL each of 6M HCl to quench the enzyme reaction at 2, 5, and 20 min. Quenched aliquots were heated at 50° C. for 40 min with 750 μL each of Ehrlich's reagent (2:24:74 w/v/v 4-(diethyl)aminobenzaldehyde: conc HCl: ethanol). Absorbance was read at 580-nm and compared with standards of serotonin (10-250 μM) or tryptamine (50 to 2000 μM).

Correction of the absorbance for dilution gives a calculated extinction coefficient of approx. 7 mM₋₁ for the serotonin-Ehrlich mixture. With tryptamine the extinction coefficient is approximately 1.4-mM₋₁.

### Example 11

Assay in of 5-HOtryptophan Decarboxylase Activity Using Ehrlich 's Reagent in Microtiter Plates

Enzyme samples (20 μL) are added to 180 μL of a cocktail containing 7.6 mL of buffer, 1 mL of 20 mM 5-hydroxytryptophan and 0.4 mL of 5 mM PLP at 37 EC. Reaction aliquots (40 μL) are transferred to a microtiter plate at 2, 5 and 10 min (target wells each containing 10 μL of 6M HCl to quench the enzyme reaction.) 200 μL of 4-(diethyl)aminobenzaldehyde solution (2:24:74 w/v/v aldehyde:conc HCl:ethanol) are added and the walls are covered. The resulting mixtures are covered and heated to 50° C. for 40 min. Absorbance of derivatized aliquots and serotonin standards (10-250 μM) is read at 580 nm.

### Example 12

Coupled Enzyme Monitoring by HPLC

To a solution containing L-serine (25 μmol), pyridoxal phosphate (0.05 μmol) and TrpBA (0.02 to 1.5 units, depending on the substrate), TDC1 (0.2 mL 0.6 units) and KPi buffer (0.05 mmol, pH 7.5) in a volume of 0.8 mL were added 200 mL of 5 mM indole derivative in 20% v/v ethanol/water. The mixture was incubated at 37° C. and aliquots were quenched and analyzed as described for Example 8. In cases where tryptamine and/or tryptophan standards were not available, peaks were collected for mass spectral analysis. Electrospray mass spectral analyses were performed.

### Example 13

Enzyme Immobilization

An enzyme solution containing 40-100 mg protein in 10 mL liquid was gently stirred with Eupergit C resin in PLP-containing buffer for 48 h at 4° C., then it was washed exhaustively to remove unbound protein. Protein loading was determined by using the Pierce bicinchoninic acid reagent (Tylliankis, P. E. et al., Anal. Biochem., 1994, 219, 335-340). Activity was determined by HPLC analysis of aliquots from reaction mixtures prepared as for the soluble enzymes, as described in Examples 8 and 12.

### Example 14

Preparation of 5-(Methoxycarbonylamino)indole (5-MCA-indole)

A suspension of 5-aminoindole HCl (1.012 g 6.0 mmol) in CH₂Cl₂ (15 mL) was cooled in an ice bath under a nitrogen atmosphere. Triethylamine (3.1 mL, 22 mmol) was added, followed by methyl chloroformate (0.75 mL, 9.7 mmol). A black precipitate formed, and stirring continued as the mixture warmed to room temperature overnight. The flask was again cooled on ice as 1 M H₂SO₄ (5 mL), CH₂Cl₂ (10 mL) and water (10 mL) were added, then the organic layer was separated and the aqueous phase was extracted with three 20 mL portions of 4:1 v/v CH₂Cl₂/EtOAc, after which the combined organic phase was washed with 10% NaHCO₃ (10 mL) and dried over Na₂SO₄. Following evaporation, the residue was chromatographed on silica gel using EtOAc/hexanes (10-35%) to give 0.540 g (47.3%) of a pale yellow oil. ¹HNMR (CDCl₃) δ 8.19(br s, 1H), 7.67 (br s, 1H), 7.26 (d, J=7.6 Hz, 1H), 7.15 (apparent t, J=2.8 Hz, 1H), 7.15 (br d, J=8.4 Hz, 1H), 6.59 (br s 1H), 6.50 (m 1H), 3.78 (s, 3H).

### Example 15

Preparation of 4-(Hydroxymethyl)-L-tryptophan (4-HOCH₂Trp)

To a solution containing L-serine (0.5 mmol), pyridoxal phosphate (0.5 μmol) and TrpBA (1 mL, 6 units) KPi buffer (0.5 mmol, pH 7.0) in a volume of 9.5 mL were added 200 mL of 50 mM indole-4-methanol in 20% v/v ethanol/water. The resulting cloudy mixture was shaken at 37° C., and the reaction was monitored by HPLC to detect the disappearance of the starting indole (ret. time 12. min) and appearance of the product. Three additional portions of the indole derivative were added at 6 h, 25 h, and 49 h, then after 75 h, the mixture was filtered and applied to a 2.5×7 cm column of Dowex 50w-X8 (H⁺ form), the column was successively washed with water (40 mL), 0.2 M sodium citrate buffer pH 3.0 (100 mL), water, 0.1 M HCl (150 mL), and water (50 mL), after which the tryptophan product was eluted with 1:3 NH₄OH_{(conc)}/water (200 mL), and 1:2:1 NH₄OH_{(conc)}/water/ethanol (250 mL). The eluate was monitored by TLC as generally described in Example 6, and the product was observed throughout the ammonia-containing washes, with TLC indicating some serine in the eluate. Rotary evaporation gave a brown residue. ESMS: 469.1 (2M+H⁺), 235.1 (M+H⁺), 217.1

### Example 16

5-(Methoxycarbonyl)amino-L-tryptophan (5-MCATrp)

5-(Methoxycarbonyl)amino-L-tryptophan was prepared as generally described in Example 15 for 4-(hydroxymethyl)-L-tryptophan, using four portions of 5-MCA-indole over a period of 16 h. HPLC analysis showed the appearance of the product (ret. time) accompanying the disappearance of the starting indole (ret. time 12. min). The mixture was applied to an ion exchange column of Dowex 50w-X8 (H⁺ form), the column was successively washed with water (70 mL), 0.1 M HCl (150 mL), and water (50 mL), after which the tryptophan product was eluted with 1:2:1 NH₄OH_{(conc)}/water/ethanol (250 mL). Rotary evaporation gave 59.6 mg of a pale green residue containing traces of serine. ESMS: 832.4 (3M+H⁺), 555.2 (2M+H⁺), 278.3 (M+H⁺).

### Example 17

Preparation of 2-Methyl-L-tryptophan

2-Methyl-L-tryptophan was prepared as generally described in Example 16 for 5-(methoxycarbonyl)amino-L-tryptophan with a total of 0.25 mmol of 2-methylindole, and the mixture was applied to an ion-exchange column and successively washed with water (30 mL), 0.5 M HCl, 0.5 M pH 3.0 Na-citrate (100 mL), water (20 mL) 1 M pH 7 KPi, before the product was eluted with 1:2:1 NH₄OH_{(conc)}/water/ethanol (200 mL). A mixture of 2-methyltryptophan and serine was obtained upon evaporation as a brown residue.

### Example 18

Enzyme Expression and Purification

All cloned enzymes were produced in E. coli by using shake flasks at 37° C. Conditions for induction of enzyme activity were optimized for each expression system. TrpBA and TnA were grown overnight in LB-amp medium (50 mg/L Na-ampicillin), then 0.2 mM β-(S-isopropyl)thiogalactoside (IPTG) was added to induce expression and cells were harvested after an additional 3 h. Clones expressing TDC1 and TDC2 were grown in LB-amp medium containing 0.2 mM IPTG at overnight. synAAAD in crude extracts had a specific activity of 0.1-0.2 units/mg and total enzyme yields of 50-100 units/L. Compared to the previously-reported clones (Moore, P. S.; Dominici, P.; Voltattorni, C. B. Biochem. J., 1996, 315, 249-256), this expression level represents a 15-30 fold improvement in total activity and approximately a 100-fold increase in specific activity. For all cloned enzymes, the major band on PAGE following DEAE cellulose chromatography was in the predicted molecular weight range, while the partially-purified pkAAAD enzyme also contained a second band of greater intensity at approximately 45 KD. Typical cell and enzyme yields are shown in Tables 1, 2, and 3.

TABLE 1
Production of tryptophan-synthesizing enzymes

| | | | | Purified |
|---|---|---|---|---|
| | Cell Yield | Protein | Crude spec | specific activity |
| Enzyme | (g/L) | (mg/mL) | activity | (Units/mg) |
| TrpBATrpBA | 7.45 | 17.9 | 1.2 | 2.2 |
| TnATnA | 7.5 | 11.9 | 0.8 | 2.0 |

Activity is expressed as μmol product formed per minute, TrpBA activity is reported for the reaction: indole+serine−>tryptophan at room temperature as described in the next section. Tryptophanase is reported for the reaction: tryptophan−>indole+pyruvate+NH₄⁺ at room temperature. The rate of tryptophan synthesis for TrpBA under our assay conditions is approximately 3-fold higher at 37° C.

TABLE 2
Production of tryptophan-decarboxylating enzymes

| | | Crude | Crude | Purified |
|---|---|---|---|---|
| | Cell Yield | protein | specific activity | specific activity |
| Enzyme | (g/L) | (mg) | (units/mg) | (Units/mg)* |
| PkAAAD | [489 g kidney | 8350 | 0.05 | 0.2 |
| | gave 380 mL | | | |
| | crude extract] | | | |
| syn AAAD | 8.0 | 805 | 0.18 | 0.3 |
| TDC-1 | 7.01 | 1048 | 0.11 | 0.3 |
| TDC-2 | 5.7 | 1121 | 0.12 | 0.2 |

Decarboxylase enzyme activity was determined using L-5-hydroxytryptophan as the substrate unless otherwise stated.

TABLE 3
Enzyme Purification

| | Crude specific | | |
|---|---|---|---|
| | activity | DEAE column | Purification factor |
| Enzyme | (units/mg) | Recovery | (−fold) |
| TrpBATrpBA | 1.2 | 80% | 1.8 |
| TnATnA | 0.8 | 92% | 2.5 |
| pkAAADpkAAA | 0.05 | 19% | 4.0 |
| syn AAAD | 0.18 | 73.1% | 1.7 |
| TDC-1 | 0.11 | 106% | 2.7 |
| TDC-2 | 0.12 | 66.0% | 1.7 |

### Example 19

Reactions Catalyzed by Tryptophan-Metabolizing Enzymes

Substituted indoles were incubated with L-serine, PLP and enzyme (TrpBA or TnA) prior to TLC analysis as generally described in Example 6. Serine, which can be used instead of pyruvate and ammonia for tryptophan synthesis with TnA, was employed as the 3-carbon acid because pyruvate tended to obscure the tryptophan product spots when plates were visualized with ninhydrin. TnA-catalyzed formation of tryptophan was also faster when serine was the 3-carbon donor. Reaction rates for several substrates were also estimated by HPLC. In several cases, HPLC-peaks were collected and submitted for mass spectral analysis, and the data is shown in Table 4.

TABLE 4
Indole substrates for tryptophan-synthesizing enzymes (TLC analysis)

| Indole Derivative | TrpBA | TnA | Product confirmation |
|---|---|---|---|
| indole | + | + | HPLC |
| 2-methyl- | − | + | HPLC |
| 4-amino- | + | + | |
| 4-nitro- | − | − | |
| 4-chloro- | − | + | |
| 4-acetoxy-[indoxyl acetate] | + | + | |
| 4-methoxy- | + | + | |
| 4-hydroxymethyl- | + | − | ESMS m/e 217(M-OH), |
| | | | 235 |
| 4-hydroxy | + | + | |
| 5-bromo- | − | + | |
| 5-chloro- | + | + | |
| 5-cyano- | − | − | |
| 5-fluoro- | + | + | ESMS m/e 223 (M + H⁺) |
| 5-hydroxy- | + | + | HPLC |
| 5-methoxy- | + | + | HPLC |
| Indole-5-carboxylic acid | − | − | |
| 5-nitro- | − | − | |
| 5-amino- | − | − | |
| 5-(methoxycarbonyl)amino- | + | − | ESMS m/e 278, 555 |
| [5-MCA-indole] | | | (2M + H) |
| 5-hydroxymethyl- | + | + | HPLC |
| 6-chloroindole | − | + | |
| 6-methoxyindole | + | (+) | |
| 6-fluoroindole | + | (+) | |
| 2,5-dimethyl- | + | − | |
| 5-methoxy-2-methyl- | + | − | |
| 5-bromoindole-2-carboxylic | − | | |
| acid | | | |
| 5,6-dimethoxy | − | (+) | |
| 5,6-methylenedioxy- | − | + | |
| 7-chloro | + | − | |

In Table 4, “+” indicates a clear ninhydrin-positive spot at the R_{f} of standard tryptophan derivatives after 16-65 h; “(+)” indicates a weak spot in the product region; “−” means no product observed, “NR” means experiment not run. ESMS=electrospray mass spectrometry. Unless otherwise indicated, the mass value given represents the singly-protonated molecular ion (M+1).

### Example 20

Substrate Ranges for AAAD and TDCs

HPLC techniques were used to monitor the decarboxylation of substituted tryptophans. Mass spectral analysis of the collected HPLC peaks was used to confirm the identity of several tryptamine derivatives, as shown in Table 5.

TABLE 5
Substrate screens for tryptophan-decarboxylating enzymes
(TLC analysis)

| Tryptophan | | | | | |
|---|---|---|---|---|---|
| Derivative | pkAAAD | SynAAAD | TDC1 | TDC2 | Product |
| L-tryptophan | (+) | NR | + | + | HPLC |
| DL-4-methyl-trp | − | + | + | + | |
| L-5-hydroxy-trp | + | + | + | ++ | HPLC |
| DL-4-fluoro-trp | + | (+) | + | + | |
| L-4-(hydroxy- | (+) | + | + | + | |
| methyl)-trp | | | | | |
| L-5-(hydroxy- | NR | − | | | |
| methyl)-trp | | | | | |
| DL-5-methyl-trp | − | − | + | + | |
| DL-5-methoxy- | NR | (+) | NR | NR | HPLC |
| trp | | | | | |
| DL-5-fluoro-trp | − | (+) | + | (+) | ESMS |
| | | | | | m/e 179 |
| L-5-(methoxy- | − | + | + | + | ESMS |
| carbonyl)-amino- | | | | | m/e 234 |
| trp (L-5-MCA- | | | | | |
| trp) | | | | | |
| DL-6-fluoro-trp | − | − | − | − | |
| DL-6-methyl-trp | − | (+) | + | + | |
| DL-7-methyl-trp | − | (+) | + | + | |
| DL-7-aza-trp | − | − | NR | NR | |

### Example 21

Relative rates for decarboxylation of L-5-hydroxytryptophan (L-5-HOtrp) and L-tryptophan were determined for TDC1 and TDC2 by use of Ehrlich's reagent, discussed in Example 10. The TDCs displayed a preference for the parent tryptophan, and lower activity was observed with L-5-HOtrp, as shown in Table 6.

TABLE 6
Relative rates for decarboxylation L-trp and L-5-HOtrp

| pH | 7.5 | 8.5 | 9.5 |
|---|---|---|---|
| ratio trp/5HOtrp TDC1 | 15.1 | 10.5 | 10.3 |
| ratio trp/5HOtrp TDC2 | 64.1 | 38.3 | 53.6 |

### Example 22

Relative Activities of Tryptophan-Synthesizing Enzymes with Indole Derivatives

For several substrates, initial rates were estimated by HPLC analysis, shown in Table 7. TnA reactions were run with L-serine as the 3-carbon substrate.

TABLE 7
Relative rates for enzymatic synthesis of tryptophan derivatives

| | Relative rate (TrpBA) | Relative Rate (TnA) |
|---|---|---|
| Indole | 100% | 100% |
| 5-MeO- | 19.0 | — |
| 2-Me- | 20.6* | — |
| 4HOCH₂— | 22.0 | — |
| 5-MCA- | 0.02* | — |
| 5-HOCH₂— | 0.03 | 1.2 |
| 5-F— | 0.19 | — |
| 5CH₃ | 0.55 | — |
| 5,6-(CH₃O)₂ | ** | 2.1 |
| 5,6-OCH₂O | 0.018 | 1.0 |
| 5-Br— | | 5.2 |

*product identity confirmed by comparison with isolated sample
**rate too slow to quantitate

### Example 23

pH-Rate Profiles for Tryptophan-Metabolizing Enzymes

All enzymes tested showed activity with L-serine and indole as substrates over a broad pH range. TrpBA displayed at least 90% of the maximum rate between pH 7.5 and 9.5, while TnA activity remained at 84% or greater than its maximum over the same range. Both AAADs TDC1 and TDC2 were active over the entire optimum range shown by TrpBA. These broad activity ranges make it clear that the coupled enzyme process is not limited by pH.

### Example 24

Coupled Enzyme Synthesis Experiments

Mixtures containing indole, L-serine, PLP and TDC1 were incubated in pH 8.5 phosphate buffer, analyzing aliquots by HPLC, as described in Example 12. Formation of both tryptophan, then tryptamine were clearly observable in a short period of time.

### Example 25

The procedure used in Example 24 was carried out by substituting indole with 2-methylindole, 4-hydroxymethylindole, 5-methoxy-, 5-hydroxymethyl-, 5-methoxycarbonyl, and 5,6-dimethoxyindole. In all cases, HPLC analysis showed a growing peak, which was assigned as the tryptophan derivative, and a second peak representing formation of tryptamine.

### Example 26

The procedure used in Example 25 is carried out to provide the corresponding tryptamine derivatives, and the products are then isolated by extraction into a suitable organic solvent, such as dichloromethane, ethyl acetate or a dialkyl ether. The solvent is removed and the products are reacted with acetic anhydride and a suitable base, such as triethylamine or potassium carbonate, to give melatonin derivatives having formula I where X is hydrogen, R₁ is NHAc and R₂ to R₅ are as set forth above.

### Example 27

The procedure used in Example 25 is carried out to provide the corresponding tryptamine derivatives, and the products are then isolated by extraction into a suitable organic solvent, such as dichloromethane, ethyl acetate or a dialkyl ether. The solvent is removed and the products are reacted with aqueous formaldehyde or a formaldehyde equivalent, such as paraformaldehyde, and a reducing agent, such as formic acid or sodium cyanoborohydride, to give N,N-dimethyltryptamine derivatives of formula I wherein X is hydrogen, R₁ N(CH₃)₂ and R₂ to R₅ are as set forth above.

### Example 28

The procedure used in Example 27 is employed, where, in place of formaldehyde, an alkyl-, aryl- or heteroalkyl or heteroaryl aldehyde or ketone is used to produce an N-mono- or N,N-disubstituted tryptamine derivative

### Example 29

A compound having the formula 1 wherein X is hydrogen, R₁ is NH₂ and R₂ to R₅ are as set forth above, produced as in Example 25, is reacted with an alkyl halide to give an N,N-dialkyl substituted tryptamine.

### Example 30

A compound having the formula 1 wherein X is hydrogen, R₁ is NH₂ and R₂ to R₅ are as set forth above, produced as in Example 25, is reacted with an alkyl bis-halide, such as 1,4-dichlorobutane, to give an N,N-cycloalkyl substituted tryptamine.

### Example 31

A compound having the formula 1 wherein X is hydrogen, R₁ is NH₂ and R₂ to R₅ are as set forth above, produced as in Example 25, is reacted with a bis-halide containing a heteroatom, such as N—, O—or S(═O)ₙ, such as bis-(2-chloroethyl)amine, to produce a substituted tryptamine where the sidechain nitrogen forms part of a heterocyclic ring containing at least one additional heteroatom.

### Example 32

The procedure used in Example 25 is carried out on indole-5-methanol to give the corresponding tryptamine derivative. Reaction with aqueous formaldehyde or a formaldehyde equivalent, such as paraformaldehyde, and a reducing agent, such as formic acid or sodium cyanoborohydride, gives 5-hydroxymethyl-N,N-dimethyltryptamine. The hydroxyl group is further functionalized by reaction with a halogenating agent, such as thionyl chloride or phosphorus tribromide, or with an active sulfonating agent, such as toluenesulfonyl chloride, methanesulfonyl chloride, or trifluoromethanesulfonic anhydride, to provide a compound of formula I wherein X is hydrogen, R₁ is NR₈R9, and R₂ to R₅ are as set forth above.

### Example 33

A compound having the formula I, X is hydrogen, R₁ is NR₈R₉, and R₂ to R₅ are as set forth above, is reacted with a nucleophile selected from nucleophilic heterocycles, nitrites, sulfide- sulfite- and busulfite ions, and sulfinyl nucleophiles.

### Example 34

The procedure of Example 33 is performed with the product of Example 32 and 1,2,4-triazole to give rizatriptan.

### Example 35

A compound having the formula I, X is hydrogen, R₁ is NR₈R9, and R₂ to R₅ are as set forth above, is reacted with a sulfinyl nucleophile composed of adducts of a mono- or dialkyl amine and sulfur dioxide (Suvorov, N. N., et. al., J. Gen Chem., U.S.S.R., 1965, 34, 1605) or a functional equivalent species [e.g. R₁R₂NH+SOCl₂+2 eq base+1 eq H₂O] to produce a dialkyltryptamine bearing a substituted alkyl group on the indole ring B.

### Example 36

The reaction of Example 35 is reproduced, except that the sulfinyl nucleophile is prepared from another S(IV) reagent, such as thionyl chloride or thionyl broimide, to produce a 3-(2-aminoethane)indolemethanesulformamide.

### Example 37

The reaction of Example 35 is reproduced, except that the sulfinyl nucleophile is prepared from methylamine and sulfur dioxide.

### Example 38

The reaction of Example 36 is reproduced, example that the sulfinyl nucleophile is prepared from at least three equivalents of pyrrolidine, thionyl chloride and one or more equivalents of water.

### Example 39

The product of Example 32 reacts with triethylammonium bisulfate in acetonitrile. The resulting sulfonic acid salt is converted to the sulfonyl chloride with thionyl chloride, then the sulfonyl chloride is allowed to react with methylamine, to give sumatriptan.

### Example 40

Immobilized tryptophan synthase, prepared by the procedure of Example 13, is agitated in suspension with a solution of an indole derivative, serine and pyridoxal phosphate. The resulting tryptophan derivative is isolated by passing the solution through a column of cation exchange resin, then eluting with a mixture of ammonia, methanol and water.

### Example 41

Tryptophan synthase and tryptophan decarboxylase are both immobilized by the procedure of Example 13 and are packed into a column. A solution containing an indole derivative, serine and pyridoxal phosphate is passed through the column at a temperature and flowrate such that the indole is completelty converted to the corresponding tryptamine is the eluate solution.

### Example 42

Organic-Aqueous Two-Phase Reactions

Indole, L-serine, PLP, TrpBA, and TDC 1 were combined as described in Example 25, 0.5 mL portions of toluene and ethyl acetate and the resulting mixtures were shaken at a rate of 200 rpm in a 37° C. incubator. Aliquots of the organic phase were removed and evaporated in a Speed-vac, then the residues were redissolved in acetonitrile-water (10% v/v) for HPLC analysis, as generally described in Example 8.

### Example 43

Directed evolution experiments may be performed with TrpBA and TDC1 in order to improve the ability of each enzyme to utilize specific substituted indoles. The redesigned TDC 1 gene serves as a starting point for directed evolution. High-throughput screening methods are applied to detect mutant enzymes with activity with non-natural substrates. Sensitivity to indole analogs and a color-indicator method for detecting the pH change accompanying the decarboxylation can be used to specifically detect reactions with non-natural substrates. High throughput assays using cell extracts and high-resolution HPLC and other analytical techniques, such as those described in Examples 6 to 13. Finally, increased quantities of available enzymes and improvements that can be realized through directed evolution will permit demonstration of the coupled enzyme system by preparation of gram quantities of several neuroactive tryptamine products. Following the coupled-enzyme indole-to-tryptamine conversion, conventional chemical modification of tryptamine primary amino groups and additional synthetic steps will be required to prepare the desired neuroactive products.

The preceding description has been presented with reference to presently preferred embodiments of the invention. As would be recognized by one skilled in the art, the inventive method described herein is in no way limited by the novel compounds also disclosed herein. Workers skilled in the art and technology to which this invention pertains will appreciate that alterations and changes in the described methods and compositions may be practiced without meaningfully departing from the spirit and scope of this invention. Accordingly, the foregoing description should not be read as pertaining only to the precise methods and compounds described, but rather should be read consistent with and as support to the following claims, which are to have their fullest and fair scope.

1. A method for producing a tryptamine derivative comprising:
contacting an indole derivative with a carboxylic acid having at least a β-carbon chain that is an α-ketoacid or a β-substituted alanine derivative in the presence of a tryptophan-synthesizing enzyme to produce a tryptophan derivative; and
contacting the tryptophan derivative with a tryptophan-decarboxylating enzyme to produce a tryptamine derivative.

2. The method of claim 1, wherein the carboxylic acid is an α-ketoacid.

3. The method according to claim 1, wherein the carboxylic acid is pyruvate.

4. The method according to claim 1, wherein the indole or indole derivative is contacted with the pyruvate in the presence of an ammonium ion source.

5. The method according to claim 1, wherein the carboxylic acid is a β-substituted alanine derivative.

6. The method of claim 1, wherein the carboxylic acid is selected from the group consisting of D-serine, L-serine, D,L-serine, O-alkyl derivatives of serine, O-acyl derivatives of serine, L-cysteine, S-alkyl derivatives of cysteine, S-acyl derivatives of cysteine, 3-halo-L-alanine derivatives, α-amino acids with a 4-carbon or longer alkyl chain that is substituted on the β-position with an oxygen, sulfur or halogen leaving group, and salts thereof.

7. The method of claim 1, wherein the carboxylic acid is D-serine, L-serine or D,L-serine.

8. The method of claim 7, wherein L-serine is produced in the enzyme reaction mixture from D-serine by action of an amino acid racemase enzyme.

9. The method of claim 1, wherein the tryptophan-synthesizing enzyme is selected from tryptophan synthase (E.C. 4.2.1.20) and tryptophanase (E.C. 4.1.99.1).

10. The method of claim 1, wherein the tryptophan-synthesizing enzyme is produced by recombinant expression in bacteria, in cultured cells of bacteria, fungi, or plants, or in a viral host.

11. The method of claim 1, wherein one or both of the enzymes is immobilized on a solid support.

12. The method of claim 11, wherein the immobilized enzyme(s) are used in a flow-reactor system.

13. The method of claim 1, wherein the indole substrate is dissolved in an organic solvent that is in contact with an aqueous solution of the carboxylic acid.

14. The method of claim 1, wherein the tryptamine derivative produced is isolated by chemoselective adsorption onto a solid surface.

15. The method of claim 14, where the adsorbing surface bears anionic groups selected from the group consisting of sulfonates, carboxylates, borates, boronates, phosphates, and phosphonates.

16. The method of claim 1, wherein the tryptophan-synthesizing enzyme is a mutant produced by a random mutagenesis technique.

17. A compound having the Formula I: 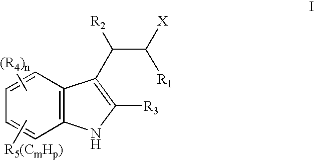
wherein:
X is hydrogen or CO₂H;
R₁ is selected from the group consisting of heterocyclic rings containing nitrogen and NR₈R₉, wherein R₈ and R₉ are each independently selected from the group consisting of hydrogen, alkyl, cycloalkyl, heterocycloalkyl, CO-R₁₀, and NHC(O)—R₁₀, wherein R₁₀ is selected from the group consisting of hydrogen, alkyl, alkoxy, cycloalkyl, aryl, and heterocyclic rings;
R₂and R₃ are each independently selected from the group consisting of hydrogen, alkyl, alkenyl, alkynyl, cycloalkyl, cycloalkenyl, cycloalkylalkyl, cycloalkenylalkyl, aryl, aralkyl, heterocyclic rings, halo, hydroxy, alkoxy, carboxy, carboalkoxy, acyloxy, cyano, nitro, acyl, acyloxyalkyl, mercapto, thioalkyl, sulfonylalkyl, sulfenylalkyl, aminoacyl, sulfonylamino, N-methylsulfonylamino, and sulfinylalkyl, or two R₄ groups together form a ring selected from the group consisting of cycloalkyl, cycloalkylalkyl, cycloalkenylalkyl, aryl, and heterocyclic rings;
each R₄ group is independently selected from the group consisting of alkyl, alkenyl, alkynyl, cycloalkyl, cycloalkenyl, cycloalkylalkyl, cycloalkenylalkyl, aryl, aralkyl, heterocyclic rings, halo, hydroxy, alkoxy, carboxy, carboalkoxy, acyloxy, cyano, nitro, acyl, acyloxyalkyl, mercapto, thioalkyl, sulfonylalkyl, sulfenylalkyl, aminoacyl, sulfonylamino, N-methylsulfonylamino, and sulfinylalkyl, or two R₄ groups together form a ring selected from the group consisting of cycloalkyl, cycloalkylalkyl, cycloalkenylalkyl, aryl, and heterocyclic rings;
R₅ is a leaving group selected from the group consisting of OH, Cl, OS(═O)₂alkyl, OS(═O)₂aryl, O—S(═O)₂O⁻, O—P(═O)(O⁻)2, O—P(═O)(O-aryl)2, O—P(═O)(O-alkyl)₂, O—P(═O)(O-alkyl)₂, O—C(═NH)alkyl, O—C(═O)H, O—C(═O)alkyl, and O—C(═O)aryl;
m ranges from 1 to 4;
n ranges from 0 to 3; and
p equals 2m or 2m−2;
wherein, when X is CO₂H, two R₄ groups do not together form a ring.

18. A compound according to claim 17, wherein R₅ is selected from the group consisting of OS(═O)₂alkyl, OS(═O)₂aryl, O—S(═O)₂O⁻, O—P(═O)(O⁻)₂, O—P(═O)(O-aryl)₂, O—P(═O)(O-alkyl)₂, O—P(═O)(O-alkyl)₂, O—C(═NH)alkyl, O—C(═O)H, O—C(═O)alkyl, and O—C(═O)aryl.

19. A compound according to claim 17, wherein R₅ is selected from the group consisting of —OS(═O)₂perfluoroalkyl, O—C(═NH)CCl₃, O—C(═O)perchloroalkyl, O—C(═O)perfluoroalkyl, and O—C(═O)-4-NO₂Ph.

20. A compound according to claim 17, wherein R₁ is selected from the group consisting of NH₂ and N(CH₃)₂.

21. A compound according to claim 20, wherein R₅ is selected from OH, OSO₂CH₃, OSO₂C₆H₅CH₃, OSO₂CF₃, and OSO₂C₆H₅NO₂.
